(19) United States
(12) Patent Application Publication
(10) Pub. No.: US 2025/0034521 A1
(43) Pub. Date: Jan. 30, 2025

(54) MAMMALIAN CELL CULTURE PROCESS FOR RECOMBINANT PROTEIN PRODUCTION

(71) Applicant: Lupin Limited, Mumbai (IN)

(72) Inventors: Sanjay Kumar Tiwari, Maharashtra (IN); Ankit Sharma, Maharashtra (IN); Snehit Shidhore, Maharashtra (IN)

(73) Assignee: Lupin Limited, Mumbai (IN)

(21) Appl. No.: 18/709,406

(22) PCT Filed: Nov. 10, 2022
(86) PCT No.: PCT/IB2022/060809

§ 371 Date: May 10, 2024

(30) Foreign Application Priority Data
Nov. 11, 2021 (IN) 202121051759

(51) Int. Cl. C12N 5/075; C07K 14/47

(52) U.S. Cl. CPC C12N 5/0609; C07K 14/473; C12N 2511/00

(57) ABSTRACT

The present invention relates to a process for producing a recombinant protein in a cell culture medium. comprising culturing mammalian cells expressing the protein of interest in a production medium at suitable conditions: and supplementing the production medium with feed components in such quantity that the residual carbon to nitrogen ratio is controlled. The present invention further relates to a process for producing a recombinant protein in a cell culture medium, wherein recombinant protein of interest has increased acidic variants and/or acidic variants and reduced basic variants.

### FIELD OF THE INVENTION

Present invention relates to the field of cell culture media and a method for culturing of mammalian cells to produce recombinant proteins. More particularly the present invention relates to increasing cell culture productivity and modulating quality of the recombinantly produced glycoproteins in terms of charge variant profile and sialylation.

### BACKGROUND OF THE INVENTION

The production of therapeutic proteins for biopharmaceutical applications typically comprises of cultivating a cell line using a media containing nutrients such as glucose, amino acids, growth factors, trace elements, salts, etc. Conventionally, the mammalian cell culture process for production of many of the glycoproteins involves controlling feeding of specific media components so as to achieve desired charge variant profile of the recombinantly produced proteins.

Charge heterogeneity of cell culture based recombinant proteins such as monoclonal antibodies, fusion proteins and peptides, is a critical attribute that can influence biological activity and safety of such proteins. Cell culture media components such as amino acids, metal ions and vitamins have great influence on charge variant profile of the recombinantly produced proteins.

Hong et al, Applied Microbiology and Biotechnology, 2014, 98(12), 5417-25 discloses that the addition of sodium butyrate to the cell culture showed increase in the basic variants profile of monoclonal antibodies. Likewise, Zhang et al, Applied Microbiology and Biotechnology, 2015, 99(16), 6643-52 discloses amino acids such as arginine and lysine inhibit carboxypeptidase enzyme which ultimately results into an increase of lysine variants in the cell culture produced monoclonal antibodies. Further, Luo et al. 2012 discloses if concentration of copper is increased in the broth along with a reduction in concentration of zinc, the lysine variants in the monoclonal antibodies to increase, which suggests that concentrations as well as ratio of copper and zinc in the cell culture process also significantly affect the charge heterogeneity in the recombinantly produced proteins. Also, higher proline amidation has been associated with higher concentration of copper. Further, Hossler et al. 2015, Biotechnology Progress 31(4), discloses that the supplementation of bioflavonoid chemical family into culture media can reduce the acidic charge variants. Niu et al. Bioresources and Bioprocess (2018) 5:42 discloses supplementation of uridine in a fed batch cell culture has greater effect on charge variant profile of monoclonal antibodies by decreasing the acidic variants. In short, the control of charge variant profile has been largely achieved by controlling feeding of specific media components such as metal ions or amino acids such or nucleosides.

While glucose is one of the most readily used carbon and energy source, lactic acid, which is often produced during the cell culture, as well as some of the amino acids could also act as carbon source under certain culture conditions. Likewise, deamination of various amino acids present into the cell culture broth results into release of ammonia which accumulates as residual ammonia and acts as an easy source of nitrogen for growth and maintenance of cells. However, excess of the residual ammonia also becomes toxic and inhibitory for growth of cells beyond a certain concentration.

However, there is need in the art to develop an improved cell culture process to produce recombinant proteins with desired charge variant profiles while retaining the productivity of protein over baseline process. The present inventors have developed a novel cell culture process to get improved productivity and control the charge variant profile of the glycosylated proteins.

### SUMMARY OF THE INVENTION

In an embodiment, the invention relates to a process for producing a recombinant protein in a cell culture medium, the process comprising culturing mammalian cells expressing the protein in a production medium at suitable conditions, wherein ratio of residual carbon to nitrogen (C/N) is increased from the baseline culture.

In another embodiment, the invention relates to a process for producing a recombinant glycoprotein in a cell culture medium, comprising:
a) culturing mammalian cells expressing the protein in a production medium at suitable conditions; and
b) supplementing the production medium with feed components, wherein feed components are added in such quantity that the residual carbon to nitrogen ratio (C/N) is controlled, and
c) isolating the protein of interest with desired charge variant profile and sialylation.

In yet another embodiment, the invention relates to a process for producing a recombinant glycoprotein in a cell culture medium with an increased productivity, the process comprising: culturing the mammalian cells expressing the protein in a production medium at suitable conditions, wherein ratio of residual carbon to nitrogen (C/N) is increased from the baseline culture.

In one more embodiment, the invention relates to a process for producing a recombinant glycoprotein having an increased sialylation, the process comprising: culturing the mammalian cells expressing the protein in a cell culture production medium at suitable conditions, wherein ratio of residual carbon to nitrogen (C/N) is increased from the baseline culture.

In one more embodiment, the invention relates to a process for producing a recombinant glycoprotein having reduced basic variants, the process comprising: culturing the mammalian cells expressing the protein in a cell culture production medium at suitable conditions, wherein ratio of residual carbon to nitrogen (C/N) is increased from the baseline culture.

### DESCRIPTION OF THE DRAWINGS

In all the figures described below, C represents carbon concentration from Glucose; and C* represents residual carbon concentration based on carbon from Glucose and Lactate; N represents residual nitrogen concentration based on ammonia.

FIG. 1 depicts residual glucose, lactate, ammonia and titer profile of a baseline cell culture process for production of VEGF Fc fusion protein carried out according to example 1.

FIG. 2 depicts C/N profile during production phase; and charge variant and total sialylated glycoform profile of VEGF Fc fusion protein at harvest stage, when carried out according to example 1.

FIG. 3 depicts level of residual glucose, lactate, residual ammonia and C/N profile in the shake flask experiment performed by targeting higher levels of carbon according to example 2.

FIG. 4 depicts titer and charge variant profile of the proteins at harvest stage, at different levels of residual carbon prepared according to example 2.

FIG. 5 depicts level of residual glucose, lactate, residual ammonia and C/N profile in the shake flask experiment performed by targeting lower levels of nitrogen according to example 3.

FIG. 6 depicts titer and charge variant profile of the proteins at harvest stage, at different levels of residual nitrogen prepared according to example 3.

FIG. 7 depicts level of residual glucose, lactate, residual ammonia and C/N profile in the bioreactor experiment performed at increased C/N ratio achieved by simultaneous increase in residual carbon level and reduced level of nitrogen, according to example 4.

FIG. 8 depicts titer and charge variant profile of the proteins at harvest stage, at increased C/N ratio to example 4.

FIG. 9 depicts charge variant profile of the VEGF Fc fusion proteins at harvest stage, when prepared according example 5, in shake flask experiment with increased feeding of glycine, according to example 5.

FIG. 10 depicts level of residual glucose, lactate, residual ammonia and C/N profile in the shake flask experiment performed at increased C/N ratio, when performed according to example 6.

FIG. 11 depicts titer, charge variant, and total sialylated glycoforms profile of the proteins at harvest stage, at increased C/N ratio to example 6.

FIG. 12 depicts level of residual glucose, lactate, residual ammonia and C/N profile in the 10 L experiment according to example 7, in comparison with the base line experiment.

FIG. 13 depicts titer profile at increased C/N ratio according to example 7 in comparison with base line process.

FIG. 14 depicts level of residual glucose, lactate, residual ammonia and C/N profile in the 10 L run with high C/N ratio process for production of Fc-Fusion according to example 8, in comparison with base line process.

FIG. 15 depicts titer and sialylation profile at increased C/N ratio according to example 8 in comparison with base line process.

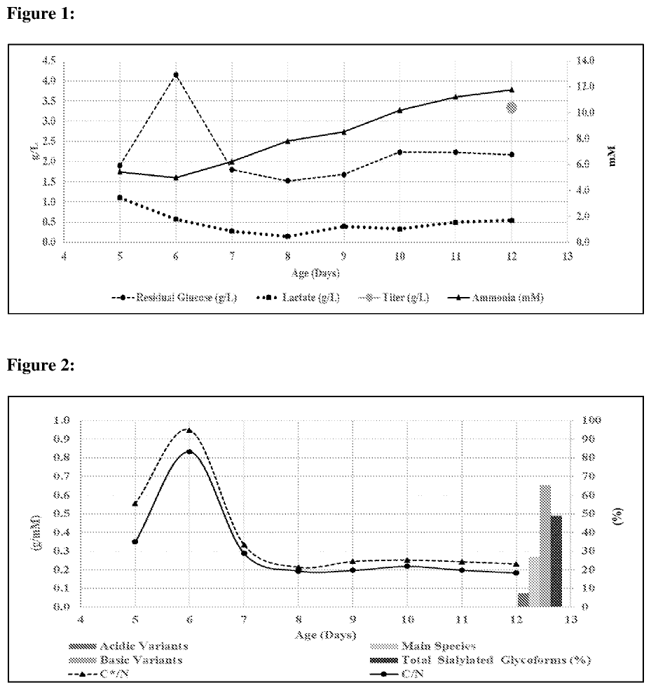

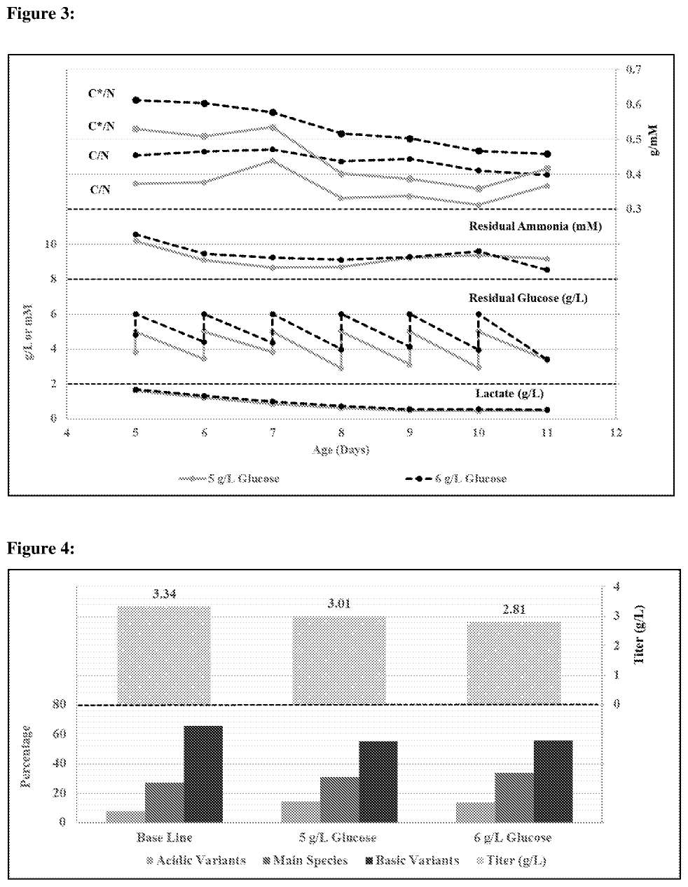

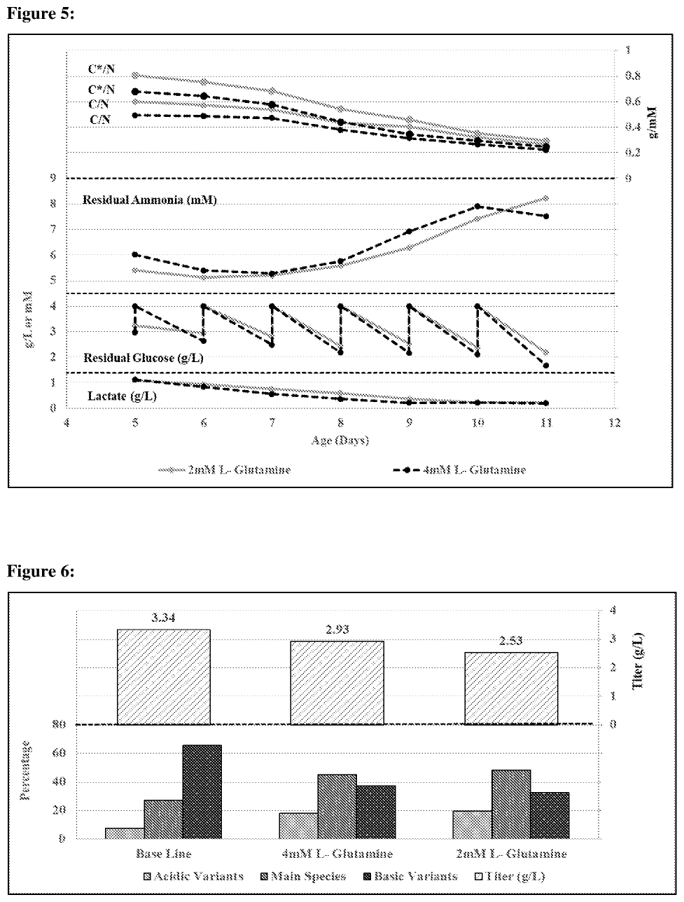

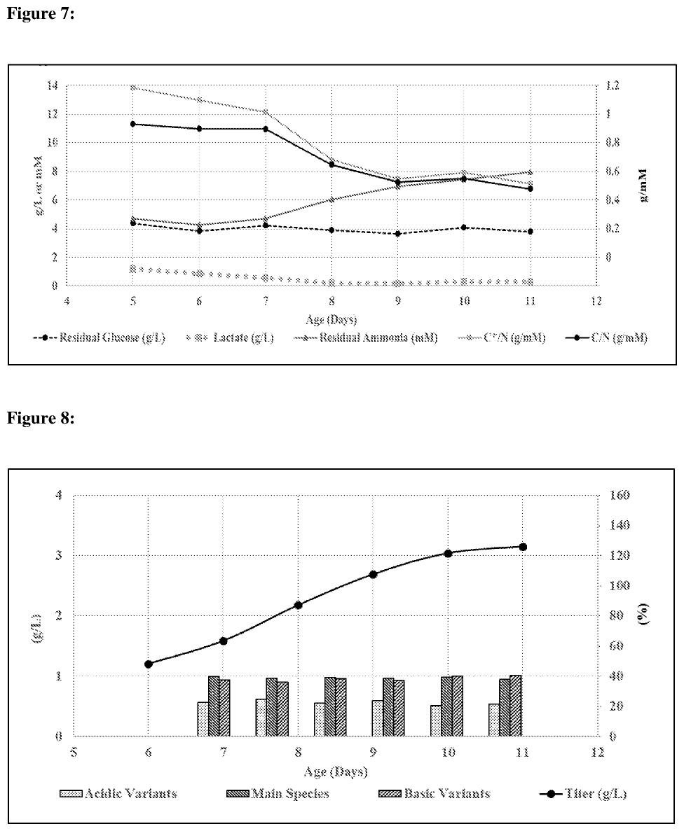

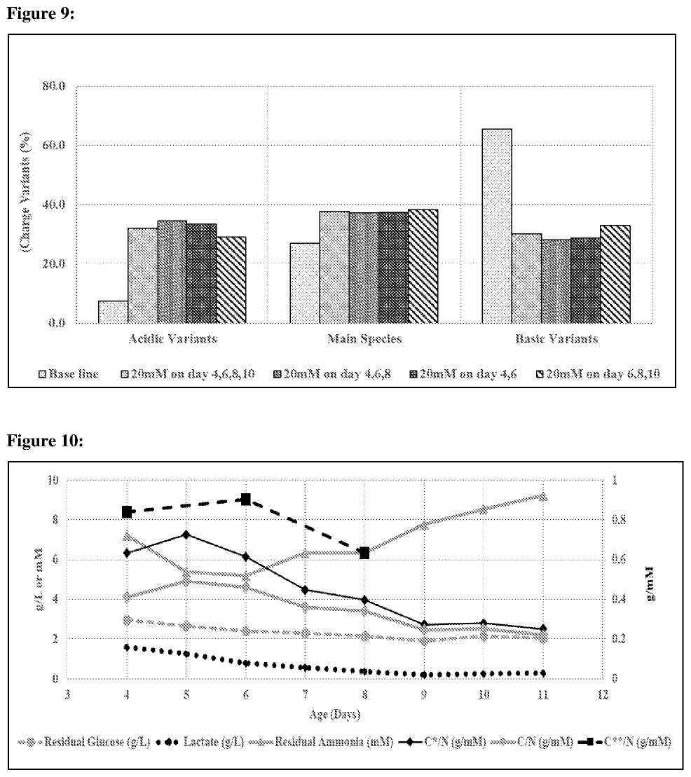

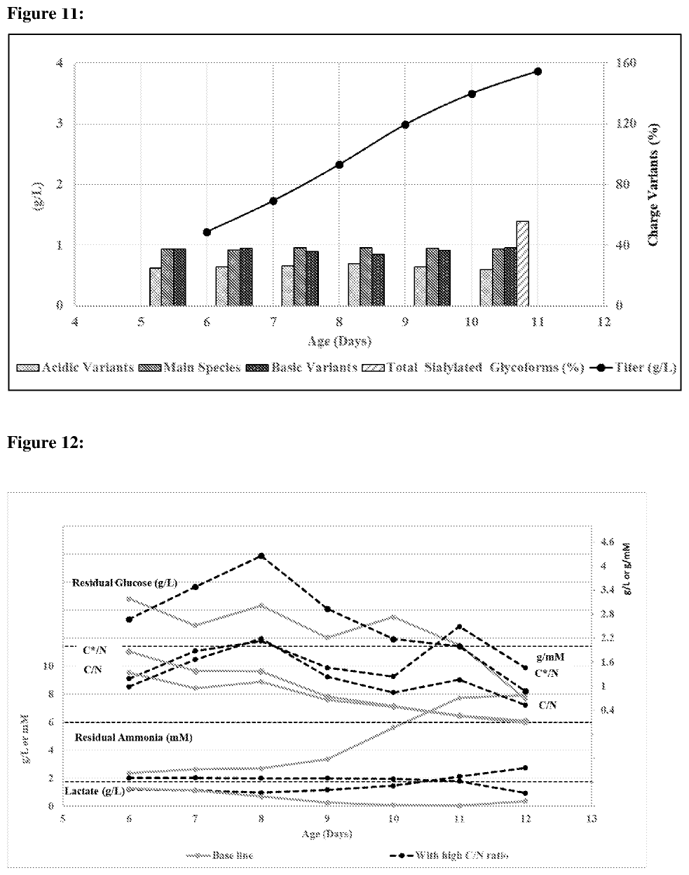

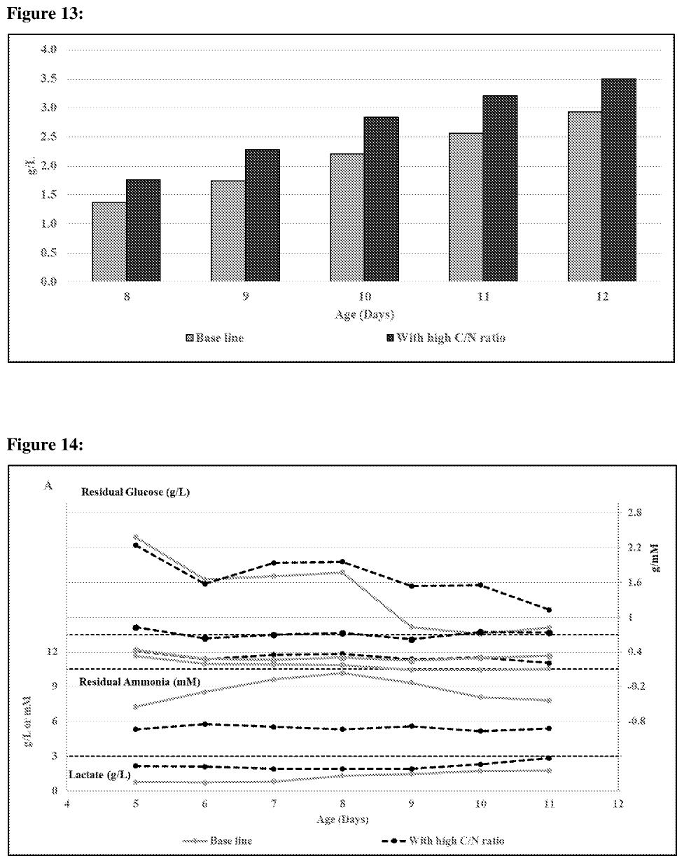

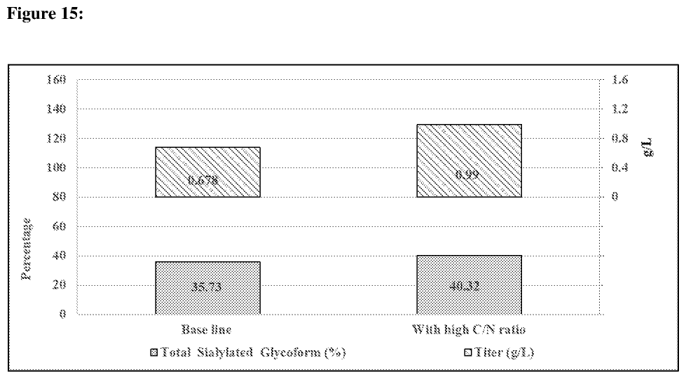

### DETAILED DESCRIPTION OF INVENTION

### Definitions

As used herein “glycoprotein” refers to one or more mammalian polypeptides that function as a discrete unit. The “glycoprotein” includes fusion proteins and monoclonal antibodies used for biopharmaceutical applications. Examples of fusion protein include but are not limited to etanercept, abatacept, alefacept, rilonacept, belatacept, aflibercept, etc. Examples of monoclonal antibodies include but are not limited to rituximab, trastuzumab, bevacizumab, adalimumab, denosumab, palivizumab, cetuximab, omalizumab, natalizumab, panitumumab, ustekinumab, ofatumumab, pertuzumab, etc.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions may be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The ‘Host cell” is genetically engineered means have recombinant DNA or RNA and expresses a gene at elevated levels or at lowered levels, or expresses a mutant form of the gene. In other words, the cell has been transfected, transformed or transduced with a recombinant polynucleotide molecule, and thereby altered so as to cause the cell to alter expression of a desired polypeptide. The conventional methods of “genetic engineering” are known in the prior art.

The “production medium” means a cell culture medium designed to be used to culture cells during a production phase.

As used herein “baseline cell culture process” means a cell culture process that serves as a defined starting process to evaluate the effect of any change. The baseline cell culture for protein of interest may contain different level of carbon source, nitrogen source and other components in such cell culture medium.

As used herein, “mammalian cell culture” refers to a mammalian cell population that is suspended in a medium under conditions suitable to survival and/or growth of the cell population. It refers to growth and propagation of mammalian cells outside of a multicellular organism or tissue. Suitable culture conditions for mammalian cells are known in the art. Mammalian cells may be cultured in suspension or while attached to a solid substrate. As used herein, mammalian cell culture process refers to the use of recombinant mammalian cell lines such as CHO DUKX-B11, CHO S, CHO K1, CHO DG44.

As used herein, “fed-batch culture” refers to a method of culturing cells in which additional components are provided to the culture at some time subsequent to the beginning of the culture process. The provided components typically comprise nutritional supplements for the cells which have been depleted during the culturing process.

As used herein “perfusion” refers to continuous flow of a physiological nutrient solution at a steady rate, through or over a population of cells.

The present invention relates to a process for culturing recombinant mammalian cells for improved production of a recombinant protein of interest, comprises steps of:
a) culturing mammalian cells expressing the protein in a production medium at suitable cell culture conditions;
b) supplementing the production medium with feed components, wherein feed components are added in such quantity that the residual carbon to nitrogen ratio (C/N) is controlled, and
c) isolating the recombinant protein of interest.

In one aspect of the invention, the carbon to nitrogen ratio is calculated based on the components that act as sources or carbon and nitrogen, which are added either extraneously or accumulate as a residual products generated during the cell culture.

The feed components acting as carbon source such as Glucose, lactate and amino acids. Further, for calculating the carbon to nitrogen ratio, lactate from extraneously added source as well as the lactate generated during the cell culture process is also considered.

In another aspect of the invention, optimum carbon to nitrogen ratio in the cell culture for producing a protein of interest is between about 0.1 to about 5 (g/mM).

In yet another aspect of the invention, optimum carbon to nitrogen ratio in the cell culture for producing a protein of interest is between about 0.3 to about 2.5 (g/mM).

In yet another aspect of the invention, optimum carbon to nitrogen ratio with respect to residual glucose to residual ammonia (C/N ratio) in production phase of glycoprotein cell culture process is between about 0.1 to about 5 (g/mM).

In one more aspect of the invention, optimum carbon to nitrogen ratio with respect to residual glucose to residual ammonia (C/N ratio) in production phase of glycoprotein cell culture process is between about 0.1 to about 1.0 (g/mM).

In one more aspect of the invention, optimum carbon to nitrogen ratio with respect to residual glucose to residual ammonia in production phase of glycoprotein cell culture process is between about 0.3 to about 0.9 (g/mM).

In one more aspect of the invention, optimum carbon to nitrogen ratio with respect to residual glucose to residual ammonia in production phase of glycoprotein cell culture process is between about 0.3 to about 1.5 (g/mM).

In one more aspect of the invention, optimum carbon to nitrogen ratio with respect to residual glucose to residual ammonia in production phase of glycoprotein cell culture process is between about 0.3 to about 1.5 (g/mM).

In another aspect of the invention, optimum carbon to nitrogen ratio with respect ‘glucose and lactate’ to ammonia (C*/N ratio) in production phase of glycoproteins cell culture process is about 0.3 to about 1.5 (g/mM).

In one more aspect of the invention, optimum carbon to nitrogen ratio with respect ‘glucose and lactate’ to ammonia (C*/N ratio) in production phase of glycoproteins cell culture process is about 0.5 to about 1.2 (g/mM).

In one more aspect of the invention, optimum carbon to nitrogen ratio with respect ‘glucose and lactate’ to ammonia (C*/N ratio) in production phase of VEGF-Fc fusion protein cell culture process is about 0.5 to about 1.2 (g/mM).

In one more aspect of the invention, the cell culture process is carried out using cell culture conditions such as temperature between 28-39° C., preferably 29-37° C., more preferably 30-35° C.; suitable air sparge and suitable pH set points, preferably about 7.0.

In yet another aspect of the invention, the basic variants in the glycoprotein obtained according to the present invention are reduced up to about 50% as compared to baseline culture process.

In one more aspect of the invention, the basic variants in the glycoprotein obtained according to the present invention are reduced up to about 40% as compared to baseline culture process.

In one more aspect of the invention, the basic variants in the glycoprotein obtained according to the present invention are reduced up to about 10% as compared to baseline culture process.

In one more aspect of the invention, the acid variants in the glycoprotein obtained according to the present invention are increased up to about 30% as compared to baseline culture process.

In one more aspect of the invention, the acid variants in the glycoprotein obtained according to the present invention are increased up to about 20% as compared to baseline culture process.

In yet another aspect of the invention, the final productivity according to the present invention is increased up to about 10% to about 30% as compared to baseline culture process.

In one more aspect of the invention, the final productivity according to the present invention is increased up to about 20% as compared to baseline culture process.

In yet another aspect of the invention, the sialylation level in the glycoprotein obtained according to the present invention is increased up to about 10% as compared to baseline culture process.

In yet another aspect of the invention, the sialylation level in the glycoprotein obtained according to the present invention is increased up to about 5% as compared to baseline culture process

The examples which follow are illustrative of the invention and are not intended to be limiting.

### Example 1: Base Line Cell Culture Process for Production of VEGF-Fc Fusion Protein

CHO-S cell line was constructed using DHFR system to grow in serum free chemically defined medium and produce VEGF-Fc fusion protein. Cells were thawed and passaged every 3-4 days, through a series of shake flasks as per requirement of seed culture. Basal medium for thaw and passaged was the same. The cultivation was carried out in incubators at 37° C. temperature and 5% CO₂ Levels. Fed batch cultivations were done in 10 L bioreactor Sartorius BDCU II. Initial volumes for shake flasks and 10 L bioreactor were kept at 70 mL and 7000 mL respectively and these were seeded at constant seeding density of 1.0-1.4×10⁶ cells/mL.

The production bioreactor was run at process temperature of 35.5° C. and the pH was controlled at 6.75 to 7.45. The dissolved oxygen was maintained preferably at 20 to 60%, more specifically at an average of 40% throughout the cell culture process. For production shake flask experimental runs, the temperature was maintained at 35.5° C. and CO₂ was maintained at 5%.

Viable cell density and viability were calculated using standard technique of trypan blue dye exclusion test using Improved Neubauer chamber or haemocytometer. The

Biochemical analysis was performed for chemistries like glucose, lactate, glutamine and ammonia using Cedex (Roche). Charge variants analysis was performed using Maurice (Protein simple). Glycan profiling was performed using NP-UPLC.

This data shows that in a baseline process for production of a VEGF-Fc fusion protein (as described in the example 1), the productivity is about 3.3 g/L in 12 days and, the basic variant at the harvest is about 65% and the sialylation level is ˜50%

### Example 2: Cell Culture Process for Production of VEGF-Fc Fusion Protein in a Media having Increased C/N Ratio by Increasing Concentration of Glucose in the Broth

In addition to the shake flask experiments as described in the example 1, in two separate experiments, glucose solution was fed to increase residual glucose level to 5 g/L and 6 g/L respectively. This leads to increase in C/N ratio in the culture media. The glycoprotein obtained from the culture shows reduction in the basic variants (about 55%) in the glycoprotein when compared with the level of basic variants (about 65%) in the glycoprotein obtained according to the baseline culture.

### Example 3: Cell Culture Process for Production of VEGF-Fc Fusion Protein in a Media Having Increased C/N Ratio by Reducing the Amount of L-Glutamine in the Cell Culture Process

In the shake flask experiments as described in the example 1, amount of L-Glutamine in the cell culture was decreased from 6 mM in control case to 4 mM and 2 mM respectively in two different experiments.

Further, the feeding of L-glutamine during the process was not performed to further control the level of ammonia during the course of cell culture.

A noticeable impact of reduced ammonia and simultaneously increased C/N was observed on the charge variant profile as shown. The percentage of basic variant could be reduced from about 65% in control to about 32% in the experimental case.

### Example 4: Cell Culture Process for Production of VEGF-Fc Fusion Protein in a Media having Increased C/N Ratio Achieved by Simultaneous Increase in the Residual Glucose and Reduction in Residual Ammonia Levels

In another 10 L bioreactor run, increase in C/N was achieved by simultaneous increase in the residual glucose and reduction in residual ammonia levels. The obtained glycoprotein profile shows that the acidic variants were relatively higher at ˜20% while the basic variants were relatively lower ˜40% throughout the bioreactor run. This demonstrates robustness of this controlling strategy for charge variant profile.

### Example 5: Cell Culture Process for Production of VEGF-Fc Fusion Protein in a Media having Increased C/N Ratio Achieved by Feeding of an Amino Acid

While in the baseline cell culture process of VEGF-Fc fusion protein, 20 mM glycine feeding is done on day 9 to 10, series of shake flask experiments were done by supplementing feed with more glycine. In first experiment cell culture media was supplemented with 20 mM glycine on day 4, 6, 8 and 10. In another experiment, cell culture media was supplemented with 20 mM glycine on day 4, 6 and 8. In yet another experiment, cell culture media was supplemented with 20 mM glycine on day 4 and 6. In one more experiment cell culture media was supplemented with 20 mM glycine on day 6, 8 and 10. The charge variant profile of the obtained glycoproteins shows that glycine which could not only act as an additional carbon/energy source but also control residual ammonia levels. The charge variant profile in the obtained glycoproteins shows that basic variant was reduced to as low as about 28% in one of the cases.

### Example 6: Cell Culture Process for Production of VEGF-Fc Fusion Protein in a Media having Increased C/N Ratio Achieved by Feeding of Glycine on Days 4, 6 and 8

Process for the production of VEGF-Fc fusion protein in a cell culture medium as described in example 5 was repeated, wherein 20 mM of glycine was supplemented to the medium on day 4, 6 and 8. This data shows that in a baseline process for production of a VEGF-Fc fusion protein (as described in the example 1), the productivity is about 3.3 g/L in 12 days and, the basic variant at the harvest is about 65% and the sialylation level is ˜50%.

Whereas, the glycoprotein obtained according to the present example, shows the basic variant could be further reduced to 36% with a proportionate increase in acid variant up to 25% and simultaneous increase in productivity and sialylation levels are about 3.87 g/L and 55%, respectively, so around 20% increment in final productivity and 5% increment in sialylation levels are observed.

### Example 7: Cell Culture Process for Production of IgG1 Antibodies in CHO-K1 Cell Line

CHO-K1 cell line was constructed using GS system to grow in serum free chemically defined medium and to produce IgG1. Cells were thawed and passaged every 3-4 days, through a series of shake flasks as per requirement of seed culture. Basal medium for thaw and passaged was the same. The cultivation was carried out in incubators at 37° C. temperature and 5% CO₂ Levels. Fed batch cultivations were performed in 10 L bioreactor Sartorius BDCU II and single use 50 L Xcellerex (GE Healthcare). Initial volume for 10 L bioreactor and 50 L Xcellerex (GE Healthcare) were 7500 mL and 35 L, respectively, at constant seeding density of 0.5×10⁶ cells/mL.

The production bioreactor was run at process temperature of 36.5° C. from day 0 to 7 and temperature shift was done from 36.5° C. to 34° C. on day 7 and the pH was controlled at 6.75 to 7.45. The dissolved oxygen was maintained preferably at 20 to 60%, particularly average 40% throughout the cell culture process.

Viable cell density and viability were calculated using standard technique of trypan blue dye exclusion test using Improved Neubauer chamber or haemocytometer. The Biochemical analysis was performed for chemistries like glucose, lactate, glutamine and ammonia using Cedex (Roche). Titer analysis was performed using protein A HPLC method.

Impact of increased C/N ratio, was studied by carrying out another experiment, wherein the cell culture conditions were kept same as the above while C/N ratio was increased by controlling the feed strategy. This data shows that in a 50 L scale baseline process for production of an IgG1, wherein the productivity is about 2.93 g/L in 12 days. Whereas in another 10 L bioreactor run, wherein C/N was increased, it showed simultaneous increase in productivity is about 3.5 g/L, i.e. around 20% increment in final productivity.

### Example 8: Cell Culture Process for Production of TNFR-Fc Fusion Protein in CHO-DG44 Cell Line

CHO-DG44 cell line was constructed using DHFR system to grow in serum free chemically defined medium and produce Fc—fusion protein. Cells were thawed and passaged every 3-5 days, through a series of shake flasks as per requirement of seed culture. Basal medium for thaw and passaged was the same. The cultivation was carried out in incubators at 37° C. temperature and 5% CO₂ Levels. Fed batch cultivations were done in 10L bioreactor Sartorius BDCU II. Initial volumes for 10L bioreactor were kept at 5900 mL respectively and seeded at constant seeding density of 1.0-1.4×10⁶ cells/mL.

The production bioreactor was run at process temperature of 34° C. and the pH was controlled at 6.6 to 7.45. The dissolved oxygen was maintained preferably at 30 to 70%, more specifically at an average of 50% throughout the cell culture process.

Viable cell density and viability were calculated using standard technique of trypan blue dye exclusion test using Improved Neubauer chamber or haemocytometer. The Biochemical analysis was performed for chemistries like glucose, lactate, glutamine and ammonia using Cedex (Roche). Titer analysis was performed with RP-HPLC. Glycan profiling was performed using NP-UPLC.

Impact of increased C/N ratio, was studied by carrying out another experiment in 10 L bioreactor, wherein the cell culture conditions were kept same as the above while C/N ratio was increased by controlling the feed strategy.

The analysis of obtained glycoproteins shows that in a baseline process for production of VEGF-Fc Fusion protein, the productivity is about 0.678 g/L in 11 days. In another 10 L bioreactor run, wherein C/N was increased, shows simultaneous increase in productivity and sialylation levels are about 0.99 g/L and 55%, respectively, so around 40% increment in final productivity and 5% increment in sialylation levels are observed.

1. A process for culturing recombinant mammalian cells for improved production of a recombinant protein of interest, comprising steps of:
a) culturing mammalian cells expressing the recombinant protein of interest in a production medium at suitable cell culture conditions; and
b) supplementing the production medium with feed components, wherein feed components are added in such quantity that the residual carbon to nitrogen ratio (C/N) is controlled, and
c) isolating the recombinant protein of interest wherein the residual carbon to nitrogen ratio (C/N) is increased from the carbon to nitrogen ratio in a baseline cell culture.

2. The process claimed in claim 1, wherein the residual carbon to nitrogen ratio (C/N) is calculated based on the components containing carbon and nitrogen source either added extraneously or generated during the cell culture process.

3. The process claimed in claim 1, wherein the residual carbon to nitrogen ratio (C/N) is between 0.1 to 5 (g/mM).

4. The process claimed in claim 1, wherein the residual carbon to nitrogen ratio (C/N) is between 0.5 to 1.2 (g/mM).

5. The process claimed in claim 1, wherein the feed component is one or more components selected from Glucose, Lactate, Glycine or Glutamine.

6. The process claimed in claim 1, wherein the isolated recombinant protein of interest has sialylation increased up to about 10% as compared to baseline culture process.

7. The process claimed in claim 1, wherein the isolated recombinant protein of interest has acidic variants increased up to about 30% as compared to baseline culture process.

8. The process claimed in claim 1, wherein the isolated recombinant protein of interest has basic variants reduced up to about 50% as compared to baseline culture process.

9. The process claimed in claim 1, wherein the improved production of recombinant protein has productivity increased up to 30% as compared to baseline culture process.

10. The process according to claim 1, wherein recombinant protein of interest is glycoprotein selected from antibodies or fusion proteins.
